(19) United States
(12) Patent Application Publication
(10) Pub. No.: US 2021/0296108 A1
(43) Pub. Date: Sep. 23, 2021

(54) ION GUIDE AND MASS SPECTROMETER

(71) Applicant: SHIMADZU CORPORATION, Kyoto-shi, Kyoto (JP)

(72) Inventors: Yusuke TATEISHI, Kyoto-shi (JP); Yoshihiro UENO, Kyoto-shi (JP); Shinji MIYAUCHI, Kyoto-shi (JP)

(73) Assignee: SHIMADZU CORPORATION, Kyoto-shi, Kyoto (JP)

(21) Appl. No.: 16/330,640

(22) PCT Filed: Oct. 11, 2016
(86) PCT No.: PCT/JP2016/080151

§ 371 Date: Mar. 5, 2019

(51) Int. Cl. H01J 49/06; H01J 49/10; H01J 49/42

(52) U.S. Cl. CPC H01J 49/063; H01J 49/42; H01J 49/105

(57) ABSTRACT

An ion guide (222) is for use in transport of an ion incident from an upstream side toward a downstream side. The ion guide includes 2n rod electrodes (n is an integral number greater than or equal to 2) equally spaced and surrounding an ion optical axis (C) that is a central axis of a flight path of the ion, a voltage applying unit (30) that applies a radio-frequency voltage to the 2n rod electrodes, and a controller (43) that controls the voltage applying unit (30). The controller (43) prompts the voltage applying unit (30) to apply a radio-frequency voltage that generates, in a space surrounded by the 2n rod electrodes, an electric field which is a 2n-multipole electric field superimposed by a higher-order electric field component than the 2n-multipole electric field.

### TECHNICAL FIELD

The present invention relates to a multipole ion guide, an example of which is a quadrupole ion guide, and further relates to a mass spectrometer equipped with the multipolar ion guide.

### BACKGROUND ART

In a mass spectrometer including an ionization chamber and an ion source provided in this chamber, ions are produced from a sample in the ion source and transported to a mass spectrometry unit disposed on the downstream side of the ionization chamber. The transported ions are then separated in accordance with their mass-to-charge ratios to measure an intensity value for each of the mass-to-charge ratios. FIG. 1 is a drawing that schematically illustrates a single quadrupole mass spectrometer which is a typical example of the conventional mass spectrometers. The quadrupole mass spectrometer includes an ion source 101, main rods 106 that separate ions in accordance with their mass-to-charge ratios, aperture plates 107, and a detector 108. This mass spectrometer further includes, between the ion source 101 and the main rods 106, ion lenses 102 and 104 that converge the ions produced in the ion source 101, and ion guides 103 and 105 that transport the converged ions to the main rods 106. The ion guide 105 disposed on the upstream side of the main rods 106 may be referred to as a pre-rod.

The ion guide 103 and the pre-rod 105 each include a multipole rod electrode, for example, a quadrupole rod electrode. FIG. 2 is a cross-sectional view of an example of the ion guide 103 including a quadrupole rod electrode. This ion guide 103 includes four rod electrodes 103a to 103d spaced at equal intervals and surrounding an ion optical axis C which is the central axis of a flight path of ions produced in the ion source 101 and directed toward the main rods 106. A radio-frequency voltage alone or a radio-frequency voltage on which a direct current voltage has been superimposed is applied to each of the rod electrodes. Radio-frequency voltages (±VcosΩt) having inverted phases are applied to adjacent ones of the rod electrodes. The direct current voltage (U) is common to all of the rod electrodes.

The mass-to-charge ratio of any ion that can pass through a space surrounded by these rod electrodes depends on voltage applied to each of the rod electrodes. A relationship between such the mass-to-charge ratio and the applied voltage may be expressed by the following formulas that define ion motion (Mathieu equation) (for example, Non Patent Literature 1).

d²x/dt²=−(2Zex/mr₀²)×(U−VcosΩt)

d²y/dt²=+(2Zey/mr₀²)×(U−VcosΩt)

where, “U” is the magnitude of a direct current voltage, “V” is the amplitude of a radio-frequency voltage, “m” is the mass of ion, “r₀” is the inradius of a rod electrode, “Ω” is the frequency of the radio-frequency voltage, “e” is an elementary charge, and “(x, y, z)” is the position of ion at time t (z axis represents the longitudinal direction of the rod electrode). Further, “Z” represents the valency of ion.

The following two parameters, a and q, are obtained by solving the Mathieu equation.

aₓ=a_{y}=8ZeU/mr₀²Ω²

qₓ=q_{y}=4ZeV/mr₀²Ω²

FIG. 3 is a drawing of a stability zone representing an ionic stability condition which is obtained from the Mathieu equation. A region defined by two axes of the parameters, a and q, represents the stability zone. When ion having a certain mass-to-charge ratio is moving in a space surrounded by the rod electrodes where a quadrupole electric field has been generated, the ion can pass through the space insofar as the coordinates (a, q) corresponding to the mass-to-charge ratio of the moving ion is included in the stability zone illustrated in FIG. 3. Otherwise, the ion diffuses in the space, failing to pass through the stability zone. In order to transport ions having a broad range of mass-to-charge ratios to the main rods 106 through the stability zone, the value of q (amplitude V of radio-frequency voltage) is so decided in the ion guide 103 and the pre-rod 105 that any ions having mass-to-charge ratios in a range of measurement target values are included in the stability zone at a=0 (direct current voltage U=0) at which the stability zone has a greatest area in the direction of q axis. FIG. 3 presents a first stability zone most often used among a plurality of stability zones that are currently present.

### CITATION LIST

### Non Patent Literature

Non Patent Literature 1: Peter H. Dawson, “Quadrupole Mass Spectrometry and its Applications”, AVS Classics in Vacuum Science and Technology (1995): P9-20, 95-119.

Non Patent Literature 2: Nikolai Konenkov, Frank Londry, Chuanfan Ding and D. J. Douglas, “Linear Quadrupoles with Added Hexapole Fields”, Journal of the American Society for Mass Spectrometry 17, 1063 (2006).

### SUMMARY OF INVENTION

### Technical Problem

The ions having a certain range of mass-to-charge ratios that are allowed to pass through the ion guide 103 and the pre-rod 105, however, may include an undesired ion(s) (interfering ion(s)) in addition to ions to be analyzed. The interfering ion(s), if included, is also transported to the downstream main rod 106. Assuming that the ion source 101 is a mass spectrometer equipped with an inductively coupled plasma ion source that ionizes a sample using inductively coupled plasma of argon gas (an inductively coupled plasma mass spectrometer, ICP-MS), ion source 101 may abundantly produce, in addition to atomic ions, argon ions and ions including atomic ion and argon (argon-attached ions). The argon-attached ions thus produced are, along with the target ions, subjected to mass spectrometry in the main rods 106 and then incident into the detector.

A detector typically used in the mass spectrometer may be a micro channel plate (MCP) detector or a secondary electron multiplier (SEM). When a scan analysis is performed with an MS equipped with an inductively coupled plasma ion source (ICP-MS) and an MCP detector, for example, abundant argon ions and argon-attached ions may be incident into the MCP detector, and a resulting flow of large current inside the detector may result in a saturation state. Then, measurement of the ions thereafter entering the MCP detector is not possible for a certain duration of time (dead time).

While the mass spectrometer equipped with the inductively coupled plasma ion source and MCP detector has been given as a specific example, a mass spectrometer used in combination with liquid chromatograph, for example, may undergo a similar problem caused by ions having a certain mass-to-charge ratio(s) that are abundantly produced from the mobile phase of the liquid chromatograph.

To address the issues of the known art, the present invention provides an ion guide that may reduce adverse impact from any interfering ions having a certain mass-to-charge ratio(s) and a mass spectrometer equipped with such an ion guide.

### Solution to Problem

An ion guide according to a first aspect of the present invention made in order to address the above issues is for use in transport of an ion incident from an upstream side toward a downstream side. The ion guide includes:

a) 2n rod electrodes (p is an integral number greater than or equal to 2) equally spaced and surrounding an ion optical axis that is a central axis of a flight path of the ion;

b) a voltage applying unit that applies a radio-frequency voltage to the 2n rod electrodes; and

c) a controller that controls the voltage applying unit, the controller prompting the voltage applying unit to apply a radio-frequency voltage that generates an electric field in a space surrounded by the 2n rod electrodes, the electric field being a 2n-multipole electric field superimposed by a higher-order electric field component than the 2n-multipole electric field.

Examples of the rod electrode may include a columnar electrode, a split rod electrode which is a columnar electrode longitudinally divided into plural pieces, and a virtual rod electrode in which plural plate-shaped electrodes are arranged along the ion optical axis. The “2n” is typically 4 (n=2), in which case an electric field, which is obtained by superimposing a higher-order electric field component than a quadrupole electric field on the quadrupole electric field, is generated in a space surrounded by four rod electrodes. A higher-order electric field component than the quadrupole electric field may be a hexapole electric field or an octupole electric field. For example, such an electric field may be generated by performing a simulation previously to decide the radio-frequency voltages to be applied to the 2n rod electrodes.

As described above, in the mass spectrometer according to the present invention, an electric field, which is obtained by superimposing a higher-order electric field component (harmonic component) than a 2n-multipole electric field on the 2n-multipole electric field, is generated in a space surrounded by the 2n rod electrodes. A potential Φ of such a multipole electric field is expressed in the formula below.

Φ(r, θ)=ΣKm·(r/R)m·cos(mθ)

where “r” and “θ” are respectively a radial position and an angle in a polar coordinate system, “Σ” is a sum of “m”, “m” is an order of the multipole electric field (harmonic component), “Km” is a harmonic component of an 2m-multipole electric field, and “R” is an inradius of the rod electrode. As for a quadrupole electric field generated in the conventional quadrupole ion guides, any terms in the formula but m=2 may be negligibly smaller than m=2.

For easy understanding of the present invention, a description is given below with reference to an example in which n=2, i.e., four rod electrodes are used. In the presence of any harmonic component in which the order “m” is greater than or equal to 3, a harmonic resonance line appears in the stability zone obtained from the Mathieu equation (for example, Non Patent Literature 1). Even in the stability zone, ion having a mass-to-charge ratio corresponding to any point on the harmonic resonance line may diffuse, resulting in a much reduction in passing efficiency of such an ion. FIG. 4 is a drawing of a stability zone in the presence of harmonic components in which the order “m” is 3, 4, 6 (equivalent to 6-, 8-, 12-multipole components). In the drawing, a broken line is a third-order resonance line, a dash-dot line is a fourth-order resonance line, and a two-dot chain line is a sixth-order resonance line. Black dots in the drawing are points of intersection of these resonance lines with a=0. A voltage to be applied to the ion guide is decided, so that the q value of interfering ion having a known mass-to-charge ratio coincides with any of these points of intersection. This may allow such an interfering ion having a certain mass-to-charge ratio to diffuse in the ion guide, decreasing the number of interfering ions introduced to the downstream side of the ion guide. The q value of interfering ion, insofar as it falls on the resonance line, may be coincident with any point but the points of intersection a=0. This may be rephrased that the radio-frequency voltage on Which a direct current voltage has been superimposed may be applied to each rod electrode.

According to the first aspect, the radio-frequency voltage to be applied to the 2n rod electrodes equally spaced around the ion optical axis are decided based on the simulation so as to generate, in the ion flight space, an electric field on which a higher-order electric field than the 2n-multipole electric field has been superimposed. As described in Non Patent Literature 2, such an electric field may be generated otherwise; by arranging the 2n rod electrodes at unequal intervals around the ion optical axis, with their positions being slightly displaced from the generating position of an ideal 2n-multipole electric field, and/or, by using 2n rod electrodes including at least one rod electrode shaped differently in cross section to the other rod electrodes.

An ion guide according to a second aspect of the present invention made in order to address the above issues is for use in transport of an ion incident from an upstream side toward a downstream side. The ion guide includes:

a) 2n rod electrodes (n is an integral number greater than or equal to 2) disposed so as to surround an ion optical axis that is a central axis of a flight path of the ion, the 2n rod electrodes being unequally spaced and/or cross-sections of the 2n rod electrodes being non-identical;

b) a voltage applying unit that applies a radio-frequency voltage to the 2n rod electrodes; and

c) a controller that controls the voltage applying unit, the controller prompting the voltage applying unit to apply radio-frequency voltages having inverted phases between adjacent ones of the 2n rod electrodes disposed around the ion optical axis.

Like the first aspect, the second aspect may allow diffusion of interfering ions having a certain mass-to-charge ratio(s) in the ion guide to decrease the number of interfering ions introduced to the downstream side of the ion guide.

A mass spectrometer according to the present invention includes:

a) an ion source that produces an ion from a sample; and

b) a mass separator that separates the ion produced in the ion source in accordance with a mass-to-charge ratio of the ion.

The mass spectrometer further includes the ion guide according to the first aspect or the second aspect interposed between the ion source and the mass separator.

### Advantageous Effects of Invention

The ion guide and the mass spectrometer according to the present invention may successfully reduce adverse impact from interfering ions having a certain mass-to-charge ratio(s).

### BRIEF DESCRIPTION OF DRAWINGS

FIG. 1 illustrates a schematic diagram of a single quadrupole mass spectrometer.

FIG. 2 illustrates a cross-sectional view of an ion guide.

FIG. 3 illustrates a graph of a stability zone representing an ionic stability condition in a space where a quadrupole electric field is generated.

FIG. 4 illustrates a graph of a stability zone representing an ionic stability condition in a space where an electric field is generated by superimposing a higher-order electric field than the quadrupole electric field on the quadrupole electric field.

FIG. 5 illustrates a diagram of principal components in an inductively coupled plasma mass spectrometer which is a working example of the mass spectrometer according to the present invention.

FIG. 6 illustrates conceptual drawings explaining removal of an ion having a certain mass-to-charge ratio in the ion guide according to the present invention.

FIG. 7 illustrates graphs of simulation results illustrating a relationship between different orders of high-order harmonic components and their ratios of superimposition on a quadrupole electric field and ion passing efficiency.

FIG. 8 illustrates graphs of a spectrum of ion passing efficiency relative to mass-to-charge ratios when the q value of an ion guide is adjusted to allow an ion having the mass-to-charge ratio of 40 to fall on a resonance line in an electric field on which a third-order harmonic component has been superimposed by 1.0%.

FIG. 9 illustrates a graph of equipotential lines in cross section orthogonal to an ion optical axis in a case where the third-order harmonic component is included by 1.0%.

FIG. 10 illustrates a diagram and graphs of a simulation result showing changes of harmonic components relative to r/r₀.

FIG. 11 illustrates diagrams and graphs of a simulation result showing changes of high-order harmonic components relative to displacements from positions of conventional rod electrodes.

FIG. 12 illustrates graphs of an ion stability zone in connection with cross-sectional shapes and positions of rod electrodes decided by the simulation.

FIG. 13 illustrates a graph of a distribution of potentials (equipotential lines) after a third-order harmonic component is superimposed by 1.0%.

FIG. 14 illustrates a diagram of decided shapes and positions of tubular rod electrodes inscribed in the equipotential lines of FIG. 13 so as to generate an electric field coincident with these equipotential lines.

FIG. 15 illustrates graphs of an ion stability zone in connection with cross-sectional shapes and positions of rod electrodes decided by another simulation.

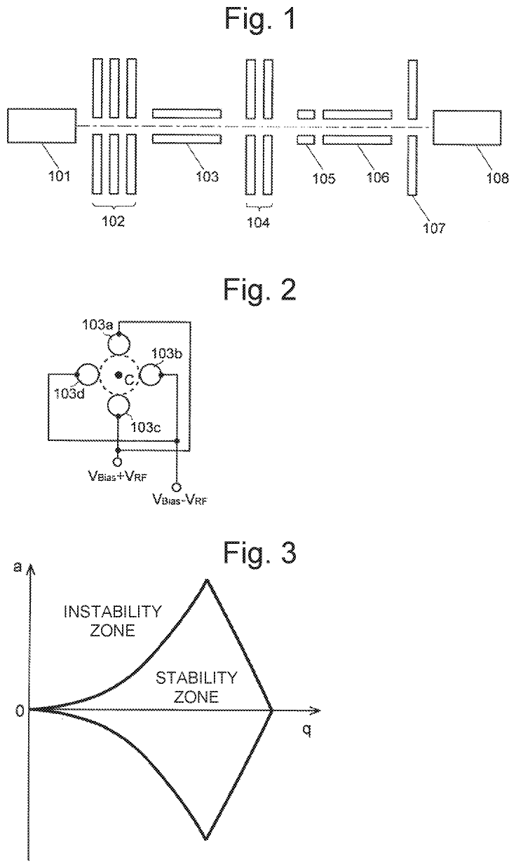

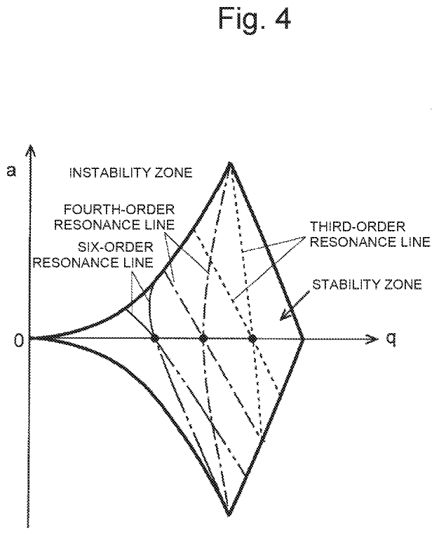

### DESCRIPTION OF EMBODIMENTS

A working example of the ion guide and the mass spectrometer according to the present invention is hereinafter described referring to the accompanying drawings. An ion guide according to the present invention is characterized to remove an interfering ion having a certain mass-to-charge ratio and included in ions produced in an ion source. Specifically, an exemplary technical concept of the ion guide according to the present invention lies in: generating a quadrupole electric field superimposed by an electric field which is a higher-order electric field than the quadrupole electric field in an internal space surrounded by the ion guide; superimposing a resonance line of a high-order harmonic component formed by the higher-order electric field component on an ion stability zone formed by the quadrupole electric field; and removing the interfering ion by arranging the certain mass-to-charge ratio on the resonance line. This embodiment provides roughly two different technical means; one is to change a voltage conventionally applied to the rod electrode, and the other is to change a conventional shape and position of the rod electrode.

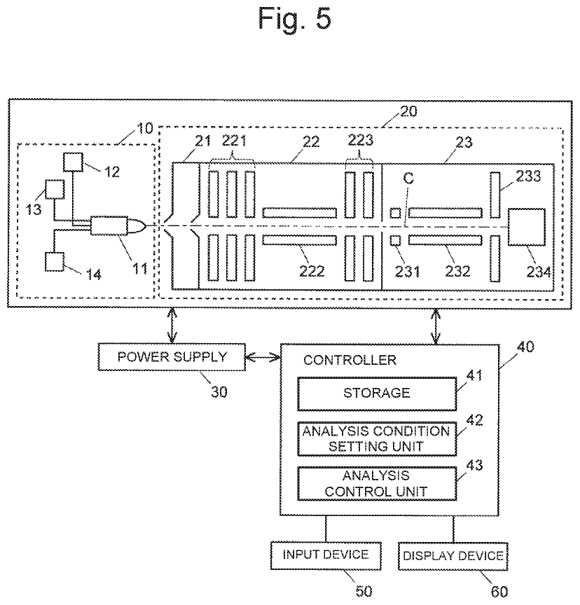

A mass spectrometer according to this working example is an inductively coupled plasma mass spectrometer equipped with an inductively coupled plasma ion source that ionizes a sample using inductively coupled plasma (ICP-SIS). As illustrated in FIG. 5, this inductively coupled plasma mass spectrometer includes, as principal components, a plasma ionizer 10, a mass spectrometry unit 20, a power supply 30, and a controller 40.

The plasma ionizer 10 includes a plasma torch 11, an autosampler 12, a nebulized gas feeder 13, a plasma gas (argon gas) feeder 14, and a cooling gas feeder (not illustrated in the drawing). The plasma torch 11 includes the following tubes formed inside; a sample gas tube for circulation of a sample gas, a plasma gas tube formed on the outer periphery of the sample gas tube, and a cooling gas tube formed on the outer periphery of the plasma gas tube. The autosampler 12 introduces a liquid sample into the sample gas tube. The nebulized gas feeder 13 feeds a nebulized gas into the sample gas tube to atomize the liquid sample. The cooling gas feeder feeds a cooling gas into the cooling gas tube.

The mass spectrometry unit 20 includes a first vacuum chamber 21, a second vacuum chamber 22, a quadrupole mass filter, and a third vacuum chamber 23. The first vacuum chamber 21 has a skimmer formed at an inlet facing an edge of the plasma torch 11. The second vacuum chamber 22 has a skimmer formed between itself and the first vacuum chamber 21 and includes first ion lenses 221, an ion guide 222, and second ion lenses 223. The third vacuum chamber 23 includes a quadrupole mass filter having pre-rods 231 and main rods 232, aperture plates 233, and a micro channel plate (MCP) detector 234. The ion guide 222 includes four rod electrodes that are arranged at equal intervals so as to surround an ion optical axis C (dash-dot line in the drawing) which is the central axis of a flight path of ions produced in the plasma torch 11 and directed toward the MCP detector 234.

The power supply 30 applies a voltage to each of the devices of the plasma ionizer 10 and of the mass spectrometry unit 20. The controller 40 includes a storage 41, and further includes, as functional blocks, an analysis condition setting unit 42 and an analysis control unit 43. A personal computer may constitute the controller 40. The functional blocks are defined and executed by prompting CPU to run a predetermined program (program for mass spectrometry). An input device 50 including a keyboard and a mouse, and a display device 60 including a liquid display are connected to the controller 40.

A storage 41 contains information of voltages to be applied to the four rod electrodes constituting the ion guide 222 for each of mass-to-charge ratios of ions. Specifically, voltage information is decided in a simulation performed earlier and then stored in the storage 41. The voltage information is used to generate, for each mass-to-charge ratio, an electric field that prevents ion having the mass-to-charge ratio from passing through the ion guide 222 but diffuses them in the ion guide 222.

When radio-frequency voltages having inverted phases are applied between adjacent ones of the four rod electrodes equally spaced and surrounding the ion optical axis C, a quadrupole electric field is generated in a space surrounded by the four rod electrodes. The mass-to-charge ratio that allows stable ion flight within the quadrupole electric field differs in accordance with the amplitude of the radio-frequency voltage applied to each of the four rod electrodes. In a case where a common direct current voltage, in addition to the radio-frequency voltages, is applied to the four rod electrodes, the mass-to-charge ratio that allows stable ion flight within the space surrounded by the four rod electrodes also changes depending on the magnitude of the direct current voltage applied.

The Mathieu equation expresses a relationship between the amplitude of the radio-frequency voltage and the magnitude of the direct current voltage, and the mass-to-charge ratio that allows stable ion flight within the space surrounded by the four rod electrodes. By solving the equation, a graph of a stability zone representing an ionic stability condition is obtained, which shows a region defined by two axes of the parameters a and q (FIG. 3). The ion guide 222 is used to let ions having a broad range of mass-to-charge ratios pass through the space and to transport them to the downstream mass separator. Normally, therefore, the q value (radio-frequency voltage amplitude V) is decided so as to place ions having a broad range of mass-to-charge ratios, which include a range of ions to be measured, in the stability zone at a=0 (direct current voltage U=0) at which the stability zone has a greatest area in the direction of “q” axis.

In this working example, to each rod electrode is applied a voltage that generates a quadrupole electric field superimposed by an electric field which is a higher-order electric field than the quadrupole electric field (harmonic component) in a space surrounded by the four rod electrodes. A potential Φ of such a multipole electric field is expressed in the formula below.

Φ(r, θ)=ΣKm·(r/R)m·cos(mθ)

where “r” and “θ” are respectively a radial position and an angle in a polar coordinate system, “Σ” is a sum of “m”, “m” is an order of the multipole electric field (harmonic component), “Km” is a harmonic component of a 2m-multipole electric field, and “R” is an inradius of the rod electrode.

In the presence of any harmonic component in which the order “m” is greater than or equal to 3, a high-order harmonic resonance line appears in the stability zone obtained from the Mathieu equation (for example, Non Patent Literature 1). Ions having mass-to-charge ratios corresponding to points on the high-order harmonic resonance line may diffuse even in the stability zone, resulting in a much reduction in passing efficiency of such ions.

The inductively coupled plasma mass spectrometer according to this working example may abundantly produce, in addition to atomic ions to be measured, argon ions and ions including atomic ion and argon (argon-attached ions). When abundant argon ions and argon-attached ions are incident into the MCP detector 234, a resulting flow of large current inside the detector may result in a saturation state. Then, measurement of the ions thereafter entering the MCP detector is not possible for a certain duration of time (dead time). In a case where mass-to-charge ratios of an ion to be analyzed and of an interfering ion are approximate to each other, there may be an overlap of the bottom of a large mass peak of the interfering ion with a mass peak of the ion to be analyzed on a mass spectrum. Then, accurate quantitative analysis may be difficult to perform. To address this issue, this working example generates, in the ion guide 222, an electric field that allows such interfering ions to be removed in the ion guide 222.

The inductively coupled plasma mass spectrometer according to this working example carries out analysis according to the following steps.

When a user inputs, through the input device 50, an instruction to start to set analysis conditions, the analysis condition setting unit 42 displays a screen on the display device 60 that allows the user to input various analysis conditions. One of the analysis conditions is the mass-to-charge ratio of an interfering ion. The mass-to-charge ratio of an interfering ion to be inputted is not necessarily limited to one value, and plural values of the ratio may be inputted.

When the user inputs analysis conditions including the mass-to-charge ratio value of an interfering ion and inputs an instruction to start analysis, the analysis control unit 43 applies voltages corresponding to the inputted analysis conditions to the devices of the plasma ionizer 10 and of the mass spectrometry unit 20. At the time, the analysis control unit 43 reads, from the storage 41, information of a voltage corresponding to the mass-to-charge ratio of the interfering ion inputted by the user, and then prompts the power supply 30 to apply the voltage to the ion guide 222.

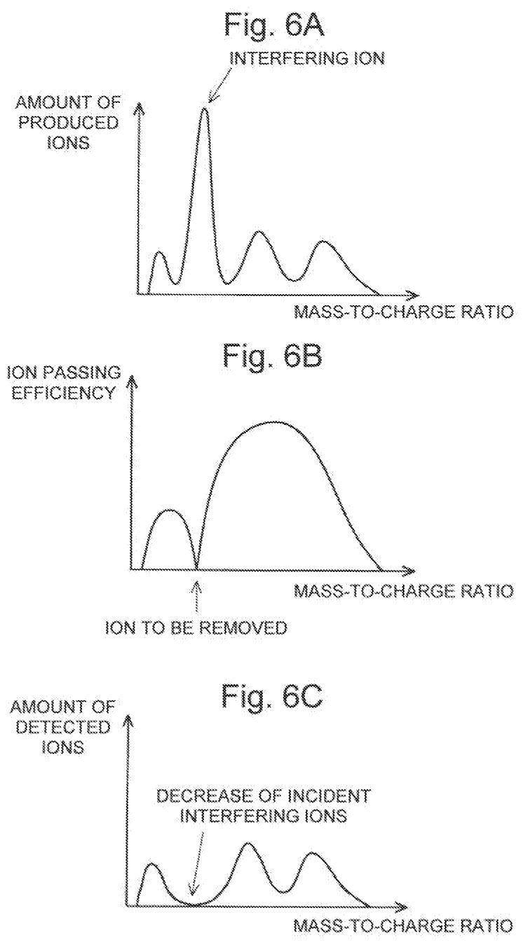

FIG. 6 illustrates conceptual drawings concerning removal of an interfering ion according to this working example. An example is discussed below, in which interfering ions having a certain mass-to-charge ratio are abundantly produced in the inductively coupled plasma ion source, as illustrated in FIG. 6A In this working example, as described earlier, an electric field, which is obtained by superimposing a higher-order electric field than a quadrupole electric field on the quadrupole electric field, is generated so as to diffuse the interfering ions in the ion guide 222 and obtain the ion passing efficiency illustrated in FIG. 6B. This may reduce the interfering ions incident into the MCP detector 234 on the downstream side of the ion guide 222, as illustrated in FIG. 6C.

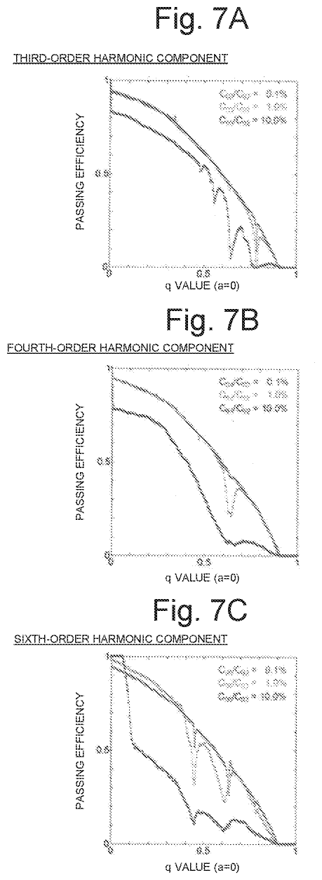

Below is described the result of a simulation performed by the present inventors in connection with electric fields of different orders superimposed on the quadrupole electric field and their ratios of superimposition. FIG. 7 illustrates graphs of simulation results showing a relationship between different orders of high-order harmonic components and their ratios of superimposition on the quadrupole electric field and ion passing efficiency, where the ion passing efficiency relative to the q value (parameter corresponding to mass-to-charge ratio) at a=0 is calculated and obtained. FIGS. 7A, 7B, and 7C respectively illustrate simulation results of a third-order harmonic component (corresponding to hexapole electric field), a fourth-order harmonic component (corresponding to octupole electric field), and a sixth-order harmonic component (corresponding to dodecapole electric field). The ion passing efficiency was calculated for the respective harmonic components, in which three different ratios of superimposition on the quadrupole electric field were used; 0.1%, 1.0%, and 10.0%.

Referring to FIG. 7A, the ratios of superimposition of the third-order harmonic component, 0.1% and 1.0%, may lower the ion passing efficiency at a certain q value (corresponding to mass-to-charge ratio). The ratio of superimposition of 1.0% may allow significant decline in the ion passing efficiency at a certain q value. The ratio of superimposition of 10%, on the other hand, may lower the ion passing efficiency at different q values. This indicates that the passing efficiencies of ions having different mass-to-charge ratios may decline at once. The ratio of superimposition of 10% may also be effective, insofar as the amplitude of the radio-frequency voltage can be decided so as to allow the mass-to-charge ratio of any interfering ion to coincide with the q values or disallow the mass-to-charge ratio of any measurement target ion to coincide with the q values.

Referring to FIG. 7B, the ratio of superimposition of the fourth-order harmonic component, 1.0%, may lower the ion passing efficiency at a certain q value. The ratio of superimposition of 0.1% may hardly affect the ion passing efficiency, while the ratio of superimposition of 10% may cause the ion passing efficiency to decline for a broad range of continuous mass-to-charge ratios. This means that the ion passing efficiency may decline not only for the interfering ion but also for other ions having mass-to-charge ratios close to that of the interfering ion. As for the fourth-order harmonic component, the ratio of superimposition should be approximately 1.0%.

Referring to FIG. 7C, the ion passing efficiency may decline at different q values when the ratios of superimposition of the sixth-order harmonic component are 0.1%, 1.0%. While decline of the ion passing efficiency at the ratio of superimposition of 0.1% is not so large similarly to FIG. 7A, a curve of the ion passing efficiency shows an abrupt drop, suggesting high selectivity of mass-to-charge ratios. The ratio of superimposition of 1.0% may relatively narrow the selectivity of mass-to-charge ratios, however, may markedly lower the ion passing efficiency in the vicinity of the certain q value. Since the ion passing efficiency may decline at once at different q values, it may be necessary for the mass-to-charge ratio of any interfering ion to coincide with these q values or for the mass-to-charge ratio of any measurement target ion not to coincide with these q values. Similarly to FIG. 7 the ratio of superimposition of 10% may cause the ion passing efficiency to decline for a broad range of continuous mass-to-charge ratios.

These results led the inventors to the conclusion that superimposing the third-order harmonic component by 1.0% may be most useful, which was discussed through another simulation.

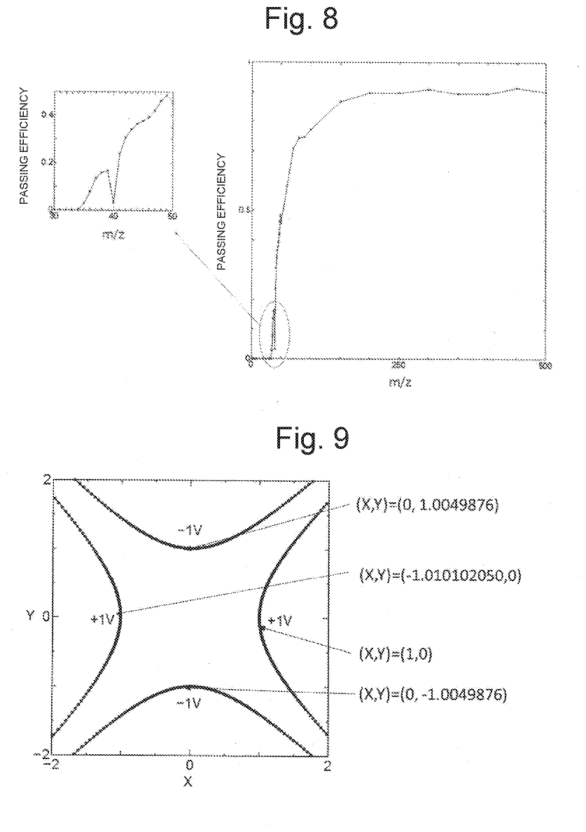

FIG. 8 illustrates graphs of a spectrum of ion passing efficiency relative to mass-to-charge ratio when the q value of an ion guide, i.e., amplitude of a radio-frequency voltage, is adjusted to allow an ion having the mass-to-charge ratio of 40 to fall on a resonance line in the case of an electric field on which a third-order harmonic component has been superimposed by 1.0%. FIG. 8 shows a sharp drop of the ion passing efficiency at the mass-to-charge ratio of 40. This figure also shows continuous change of the ion passing efficiency in other mass-to-charge region. These indicates high selectivity of mass-to-charge ratios and very little effect on ions having adjacent mass-to-charge ratios. Such lower ion passing efficiency with smaller mass-to-charge ratios may attribute to the fact that mass-to-charge ratios in this region are near an edge part of the stability zone, with no relevance to superimposition of the high-order harmonic component.

FIG. 9 illustrates a graph of equipotential lines in cross section orthogonal to the ion optical axis C when the third-order harmonic component is included by 1.0%. This graph was obtained by imparting several percentages of nonlinearity to an ideal bipolar field. An electric field having such equipotential lines, if successfully generated, may allow selective removal of interfering ions having the mass-to-charge ratio of 40.

In the earlier example, the electric field illustrated in FIG. 9 was generated by deciding amplitude of radio-frequency voltage (and a magnitude of direct current voltage) to be applied to each of the four rod electrodes equally spaced around the ion optical axis C. Such an electric field may be obtained by changing the conventional positions and shapes of the rod electrodes.

The present inventors employed two methods described below to perform a simulation relating to positions and shapes of the rod electrodes that allow the generation of an electric field including the third-order harmonic component by 1.0%.

One of them is to change by degrees positions and shapes of the tubular rod electrodes arranged, as in the known art, at equal intervals around the ion optical axis C to see if there are any resulting changes of the high-order harmonic components.

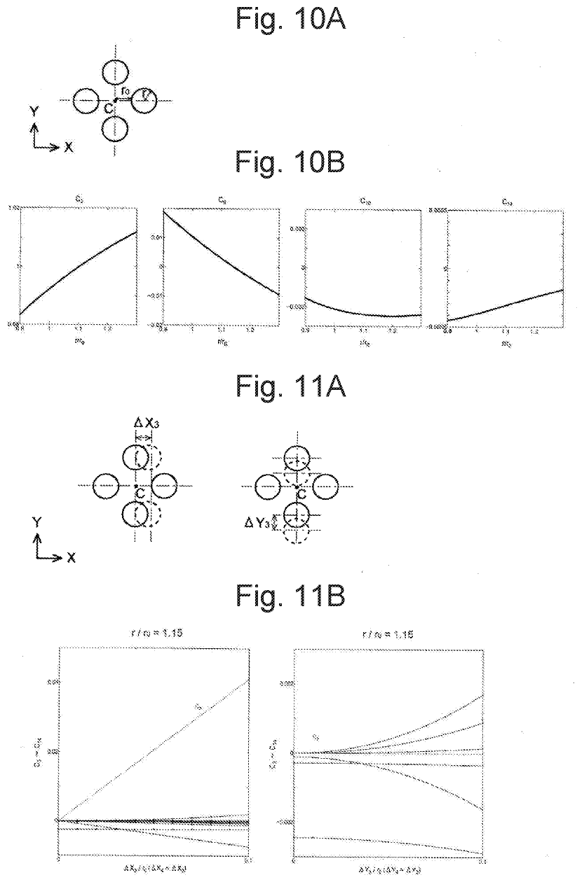

First, changes of the high-order harmonic components relative to r/r₀ were simulated, where “r” is a radius in cross section of the tubular rod electrode, and “r₀” is a distance from the ion optical axis C to the rod electrode (FIG. 10A), in order to decide an initial arrangement for simulation (conventional arrangement for the generation of a quadrupole electric field). FIG. 10B shows an obtained simulation result. In the quadrupole arrangement, because of its symmetry, harmonic components of any orders (n) but n=4m+2 (m is a natural number) are all zero. Contribution of the harmonic component to a potential distribution is proportional to the (n)th power of the radius vector r₀. A sixth-order harmonic component (C₆) is, therefore, considered to appear to provide a greatest contribution. In view of these factors, C₆=0 was defined for approximation to a most ideal quadrupole electric field, and r=1.15r₀ was also defined, where “r” was a rod diameter,

Then, changes of the high-order harmonic components relative to displacements from the initial arrangement of rod electrodes were identified through a simulation. As illustrated in FIG. 11A, ΔX₃ is an amount of movement in the direction of X axis (lateral direction in the diagram), and ΔY₃ is an amount of movement in the direction of Y axis (vertical direction in the diagram). FIG. 11B shows an obtained simulation result. The simulation result teaches that a target electric field (electric field on which the third-order harmonic component C₃ has been superimposed by 1.0%) may be obtained as a result of rod displacement in the X direction (lateral direction in the diagram) (ΔX₃=0.022r₀). On the other hand, any displacement in the Y direction only resulted in increases in the other components, with substantially no change of the third-order harmonic component.

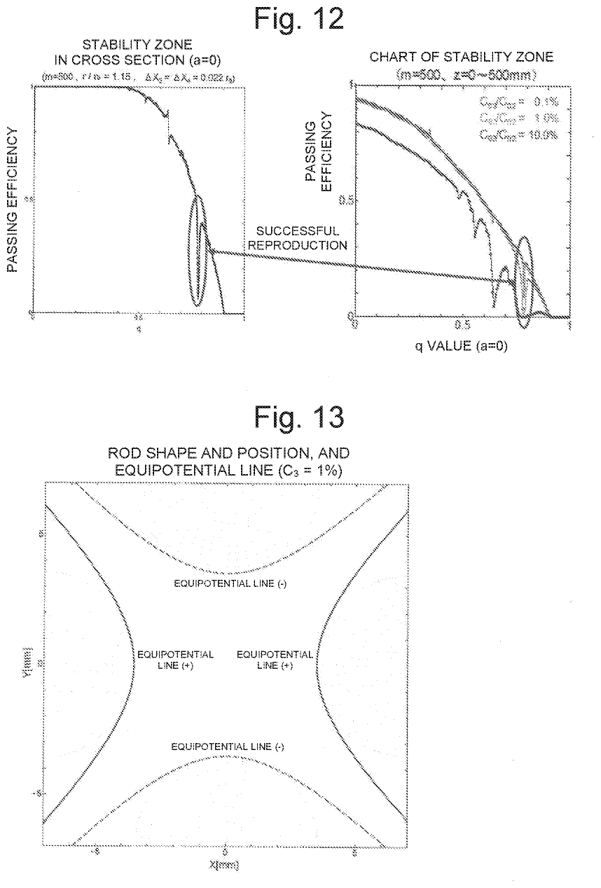

In the left-side graph of FIG. 12 is shown a calculation result of an ion stability zone when the rod electrodes were shaped in cross section as (r/r₀=1.15) and arranged as (ΔX₃=0.022r₀) which were decided as described earlier. This graph demonstrates successful reproduction of the superimposition result of the third-order harmonic component by 1.0%, as shown in the right-side graph (FIG. 7A) of FIG. 12.

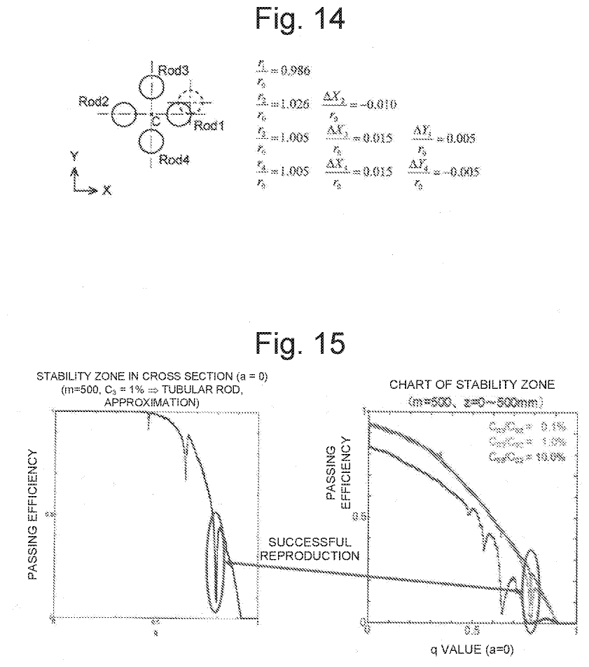

Next, the other method is described below. This method starts with drawing target equipotential lines on which the third-order harmonic component has been superimposed by 1.0% and decides positions and shapes of tubular rod electrodes inscribed in the equipotential lines so as to generate an electric field approximate to the equipotential lines. FIG. 13 illustrates a graph of a potential distribution (equipotential lines). FIG. 14 illustrates a diagram showing positions and shapes of the tubular rod electrodes decided by this method. In FIG. 14, r₀ is a distance from the ion optical axis C to the rod electrode, r₁ to r₄ are radiuses of the rod electrodes, and ΔXₖ and ΔYₖ (k=1 to 4) are displacements of the rod electrodes.

According to the described method, shapes in cross section and positions of the rod electrodes were decided as follows; shapes (r₁/r₀=0.986, r₂/r₀=1.026, r₃/r₀=1.005, r₄/r₀=1.005), and positions (ΔX₁=0, ΔX₂=−0.010r₀, ΔX₃=0.015r₀, ΔX₄=0.015r₀, ΔY₁=0, ΔY₂=0, ΔY₃=0.005r₀, ΔX₄=−0.005r₀). In the left-side graph of FIG. 15 is shown a calculation result of an ion stability zone when these positions and shapes were used. This graph also demonstrates successful reproduction of the superimposition result of the third-order harmonic component by 1.0%, as shown in the right-side graph (FIG. 7A) of FIG. 15.

The working example was described thus far by way of example, and various modifications may be made to this embodiment within the technical scope of the present invention.

While the working example has been described in connection with the inductively coupled plasma mass spectrometer, the present invention may be applicable to any other suitable mass spectrometers.

While the ion guide described in the working example includes four rod electrodes, other ion guides may be used that include rod electrodes provided in an even number of six or more. The technical features described in the working example in connection with the ion guide may also be applicable to the pre-rods of the quadrupole mass filter. Examples of the rod electrode may include a columnar electrode having any shape but circular shape in cross section, a split rod electrode which is a columnar electrode longitudinally divided into plural pieces, and a virtual rod electrode in which plural plate-shaped electrodes are arranged along the ion optical axis C. The numerical values Oven in the working example are only a few examples used to describe the specific embodiment and may be suitably changed for any apparatus actually used.

### REFERENCE SIGNS LIST

10 . . . Plasma Ionizer
11 . . . Plasma Torch
12 . . . Autosampler
13 . . . Nebulized Gas Feeder
14 . . . Plasma Gas Feeder
20 . . . Mass Spectrometry Unit
21 . . . First Vacuum Chamber
22 . . . Second Vacuum Chamber
221 . . . First Ion Lens
222 . . . Ion Guide
223 . . . Second Ion Lens
23 . . . Third Vacuum Chamber
231 . . . Pre-rod
232 . . . Main Rod
233 . . . Aperture Plate
234 . . . MCP Detector
30 . . . Power Supply
40 . . . Controller
41 . . . Storage
42 . . . Analysis Condition Setting Unit
43 . . . Analysis Control Unit
50 . . . Input Device
60 . . . Display Device

1. An ion guide for use in transport of an ion incident from an upstream side toward a downstream side, the ion guide comprising:
an analysis condition setting unit that sets a mass-to-charge ratio of an ion to be removed from the ion guide based on an input by a user;
2n rod electrodes (n is an integral number greater than or equal to 2) equally spaced and surrounding an ion optical axis that is a central axis of a flight path of the ion;
a voltage applying unit that applies a radio-frequency voltage to the 2n rod electrodes; and
a controller that controls the voltage applying unit, the controller prompting the voltage applying unit to apply a radio-frequency voltage that generates an electric field in a space surrounded by the 2n rod electrodes, the electric field being a 2n-multipole electric field superimposed by a higher-order electric field component than the 2n-multipole electric field,
wherein the higher-order electric field component removes from the space an ion having the mass-to-charge ratio set by the analysis condition setting unit.

2. An ion guide for use in transport of an ion incident from an upstream side toward a downstream side, the ion guide comprising:
an analysis condition setting unit that sets a mass-to-charge ratio of an ion to be removed from the ion guide based on an input by a user;
2n rod electrodes (n is an integral number greater than or equal to 2) disposed so as to surround an ion optical axis that is a central axis of a flight path of the ion, the 2n number of rod electrodes being unequally spaced and/or cross-sections of the 2n rod electrodes being non-identical;
a voltage applying unit that applies a radio-frequency voltage to the 2n rod electrodes; and
a controller that controls the voltage applying unit, the controller prompting the voltage applying unit to apply radio-frequency voltages having inverted phases between adjacent ones of the 2n rod electrodes disposed around the ion optical axis,
wherein an electric field generated in a space surrounded by the 2n rod electrodes includes a higher-order electric field component than a 2n-multipole electric field that removes from the space an ion having the mass-to-charge ratio set by the analysis condition setting unit.

3. (canceled)

4. The ion guide according to claim 1, wherein
the 2n-multipole electric field is a quadrupole electric field, and
the higher-order electric field component includes one of a hexapole electric field component, an octupole electric field component, and a dodecapole electric field component.

5. A mass spectrometer, comprising:
an ion source that produces an ion from a sample; and
a mass separator that separates the ion produced in the ion source in accordance with a mass-to-charge ratio of the ion,
the mass spectrometer further comprising the ion guide according to claim 1 interposed between the ion source and the mass separator.

6. The mass spectrometer according to claim 5, comprising an inductively coupled plasma ion source that ionizes a sample using inductively coupled plasma.

7. The ion guide according to claim 2, wherein
the 2n-multipole electric field is a quadrupole electric field, and
the higher-order electric field component includes one of a hexapole electric field component, an octupole electric field component, and a dodecapole electric field component.

8. A mass spectrometer, comprising:
an ion source that produces an ion from a sample; and
a mass separator that separates the ion produced in the ion source in accordance with a mass-to-charge ratio of the ion,
the mass spectrometer further comprising the ion guide according to claim 2 interposed between the ion source and the mass separator.
